(19) United States
(12) Patent Application Publication
(10) Pub. No.: US 2018/0028103 A1
(43) Pub. Date: Feb. 1, 2018

(54) A BLOOD LANCET UNITARILY-MOULDED WITH AN ELASTIC MEMBER

(71) Applicant: Chee Yen LIM, Singapore (SG)

(72) Inventors: Chee Yen LIM, Singapore (SG); Yong Wei CHIA, Singapore (SG); Wei Siong LEE, Singapore (SG)

(21) Appl. No.: 15/547,532

(22) PCT Filed: Feb. 11, 2015
(86) PCT No.: PCT/SG2015/050021

§ 371 Date: Jul. 31, 2017

(51) Int. Cl. A61B 5/151; A61B 5/15

(52) U.S. Cl. CPC A61B 5/15117; A61B 5/150022; A61B 5/15113; A61B 5/15144; A61B 5/150717

(57) ABSTRACT

The present invention relates to a blood lancet used in capillary blood sampling, in which the blood lancet is unitarily moulded with an elastic member for providing actuation force. In a preferred embodiment, the blood lancet (100) comprises a needle component (120) having a forward end (122) and a rearward end (124), an elastic member (140) disposed in the rearward end (124), and a detachable safety cap (160) disposed at the forward end (122), both of which are unitarily moulded to the needle component (120). In a second preferred embodiment, a two-part safety lancet is provided, comprising a blood lancet (1100) and a holder (1200), the blood lancet (1100) further comprising a needle component (1120) having a forward end and a rearward end, an elastic member (1140) disposed at the rearward end, a detachable safety cap (1160) disposed at the forward end, a pair of latch hooks (1170a) attached to the needle component (1120), a U-shape button (1180) having a pair of arms, each arm further comprises a hammer (1210a) and a reverse hook (1190a) at the end of the arm, wherein all the components, i.e. the elastic member (1140), the detachable safety cap (1160), the pair of latch hooks (1170a), the U-shape button (1180), the pair of hammers (1210a) and the pair of reverse hooks (1190a), are unitarily moulded on the needle component (1120).

### BACKGROUND OF THE INVENTION

### Field of the Invention

This invention relates generally to blood lancet used for capillary blood sampling. More particularly, the present invention relates to the design, the fabrication method and the use of a blood lancet unitarily moulded with an elastic member for capillary blood sampling.

### Background of the Invention

Blood extraction is required for carrying out various in-vitro diagnostic tests. There are in general two types of blood extractions: (1) the first type is the venous blood extraction (venepuncture) in which a cannula is inserted into a superficial vein to draw venous blood directly from the vein, the blood volume is normally in terms of millilitres; (2) the second type is capillary blood sampling in which a blood lancet is normally used for making an incision on a fingertip for extracting capillary whole blood from the capillary network right below the skin. The blood volume obtained is normally in the range of 0.3 ul-100 ul, which is suitable for point-of-care diagnostic applications, for instance, blood glucose monitoring, rapid lateral-flow assay tests, and other point-of-care tests. Normally, venous blood is collected at the hospital when a large blood volume is required for multiple blood tests to be carried out at the central laboratories. On the other hand, capillary blood is collected from fingertips or other skin sites with rich capillary network at home or a point-of-care for rapid blood screening tests or other specific point-of-care blood tests.

The present invention relates to the capillary blood sampling for in-vitro diagnostic blood tests. For capillary blood sampling via a finger prick, the current practice is to actuate a blood lancet with a metal spring for rapidly and linearly incising the skin to minimize the pain. The incision involves linearly inserting and retracting a needle at high speed, and the incision depth ranges from 0.5 mm to 2 mm deep. The incision (penetration and retraction) speed provided by the spring is in the range of 1 m/s-6 m/s. Therefore, for a penetration of 0.5 mm at the speed of 1 m/s, the time of penetration is 0.5 msec and the time of retraction is also 0.5 msec, giving a total of 1 msec. In addition to great reduction of the incision time, the linear and rapid incision stroke also reduces much pain by minimally disrupting the nerve ending. Hence, it is essential that a metal spring is used together with a blood lancet for effective capillary blood sampling.

Blood lancets are used in two groups of users, the first group consists of the home users who regularly sample their blood for blood tests, for instance the diabetic patients who are required to monitor their blood glucose levels daily and regularly; the second group consists of medical personnel who sample blood for patients, for instance the nurses in the hospitals.

For the home users, the blood lancets are loaded in a re-usable spring-operated lancing device so that the disposable components are minimized to save costs. For the hospital users, the spring and the blood lancet are housed in a casing (this device is called safety lancet hereinafter) so that the needle is not exposed to prevent finger-stick and that the entire device can be disposed of to prevent cross infection between patients. As a result, the safety lancet is much more expensive compared to the home-use blood lancets in long term, mainly due to the spring, which is almost universally made of metal such as stainless steel (of course there are other components such as the casing which adds on to the costs, but their contribution will be minor).

It is apparent that for the benefit of re-using the spring, the home users are required to handle the needles every time they load the blood lancet into the lancing device, for which they are exposed to finger-stick hazard. On the other hand, the hospital users throw away the spring after each use, this makes the blood sampling cost very expensive. If a home user were to use safety lancets, the costs may be doubled in the long term.

Hence, the present invention's primary objective is to reduce the cost of the metal spring by unitarily moulding an elastic member onto a blood lancet, wherein the elastic member acts to provide the required actuation force for skin incision when charged and discharged (i.e. compressed to store potential energy, then released to actuate the blood lancet).

### Closest Prior Art

U.S. Pat. No. 8,652,158 B2 to Owen Mumford Limited reported a blood lancet which is integrally moulded with a pair of undulating plastic webs (springs) at the forward end of the blood lancet. The undulating plastic webs were incorporated for returning the blood lancet after actuation. The springs in this patent do not provide any actuation that is needed for skin penetration, so a metal spring is still needed in the device.

U.S. Pat. Nos. 5,628,765 B2 and 5,755,733 B2 to APLS Co. Ltd. reported a safety lancet comprising a plastic spring member coupled to a blood lancet. Although the plastic spring member is injection moulded, it is not unitarily moulded to the blood lancet, therefore requiring two separate injection moulding processes.

### SUMMARY OF THE INVENTION

The present invention provides a blood lancet unitarily moulded with an elastic member for capillary blood sampling. In the preferred embodiment, a blood lancet 100 comprises a needle component 120 having a forward end 122 and a rearward end 124, the forward end 122 is disposed with a detachable safety cap 160 for protecting the needle tip, the rearward end 124 is disposed with an elastic member 140 for providing actuation force to the needle component 120 for skin penetration, wherein the detachable safety cap 160 and the elastic member 140 are unitarily moulded to the needle component 120. In the second preferred embodiment, a two-part safety lancet 1000 is provided. The two-part safety lancet 1000 comprises a holder 1200 and a blood lancet 1100, the blood lancet 1100 comprises a needle component 1120 having a forward end and a rearward end, a detachable safety cap 1160 disposed at the forward end, an elastic member 1140 disposed at the rearward end, wherein the detachable safety cap 1160 and the elastic member 1140 are unitarily moulded to the needle component 1120. In operation, the blood lancet 1100 is inserted into the holder 1200 which provides the necessary latching mechanism for charging and discharging the elastic member 1140 for providing actuation force to the needle component 1120.

### BRIEF DESCRIPTION OF THE FIGURES

FIG. 1 illustrates a perspective view of a blood lancet unitarily moulded with an elastic member

FIG. 2 illustrates an exploded view of a blood lancet unitarily moulded with an elastic member

FIG. 3 illustrates a perspective view of two other possible designs of blood lancet unitarily moulded with an elastic member

FIG. 4 illustrates a perspective view of a safety lancet configuration which comprises a blood lancet unitarily moulded with an elastic member

FIG. 5 illustrates an exploded view of a safety lancet configuration which comprises a blood lancet unitarily moulded with an elastic member

FIGS. 6(a) and (b) illustrate the assembly process of the safety lancet comprising a blood lancet unitarily moulded with an elastic member

FIG. 7(a) to (e) illustrate the operation of the safety lancet comprising a blood lancet unitarily moulded with an elastic member

### DESCRIPTION OF THE INVENTION

### Detailed Description of the Invention

The present invention is provided to reduce the device cost of capillary blood sampling by integrating the function of the metal spring into the blood lancet. This integration can be achieved by over-moulding an elastic member behind the needle to provide the required actuation force. A typical capillary blood sampling device can be seen in the closest prior arts '765 and '733 of APLS Co., Ltd, where a safety lancet with a plastic spring is provided. In this device, a blood lancet with which its tip is over-moulded with a detachable safety cap was incorporated to improve a previous design by Danish Design Patent MR 0933 which used a bare blade. It is non-obvious that the elastic member can be unitarily moulded to the blood lancet for providing the actuation force, as shown by the present invention.

It is a vital aspect of current capillary blood sampling that the use of a metal spring or any other spring provides a rapid and linear incision to minimize pain. As mentioned previously, the total incision time by a spring-assisted blood sampling is in the range of 1 millisecond. These characteristics greatly reduce the pain incurred if compared by a hand prick without using a metal spring. Currently, the metal spring is incorporated into a spring-operated lancing device for home use so as to minimize the blood sampling costs. This is a common practice for diabetes patients where they need to perform a few finger pricks daily to monitor their glucose levels. However, this approach subject the users to finger-stick hazard since they will need to handle the needles when loading and unloading of the lancets on/from the lancing device. There is a long-felt need to eliminate the finger-stick hazard by providing low-cost disposable safety lancets to make capillary blood sampling more viable.

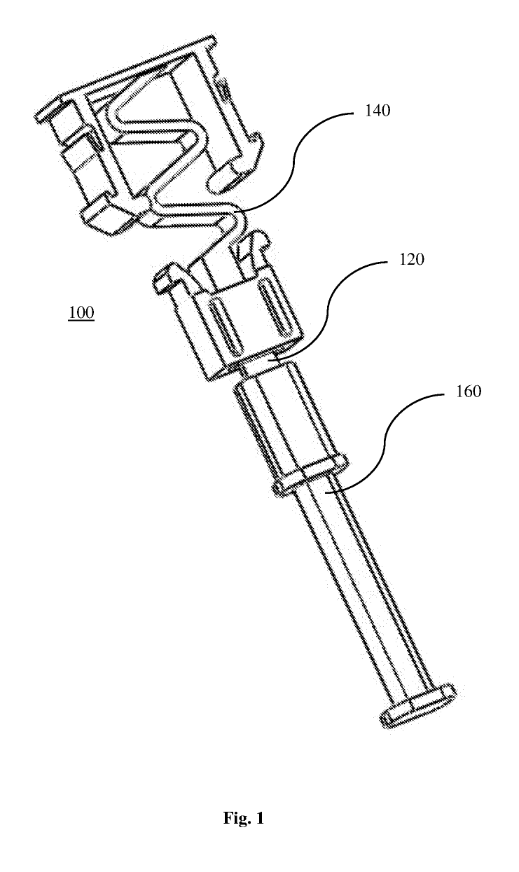

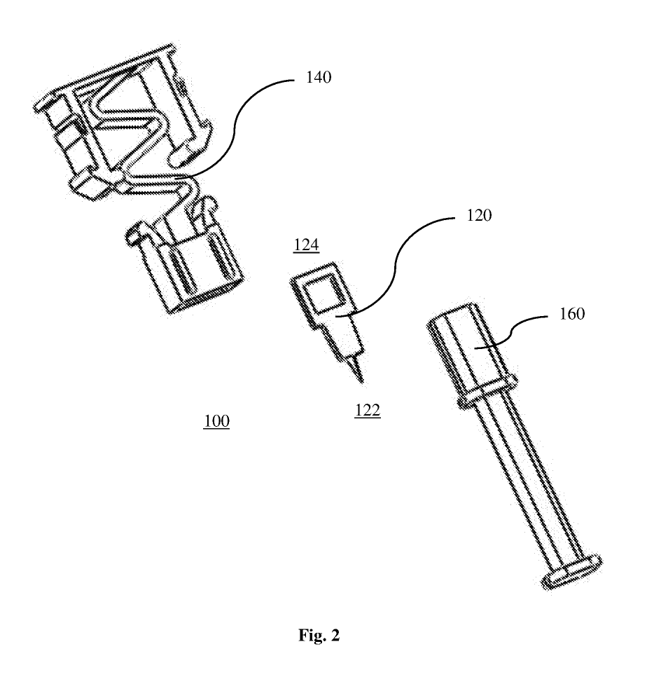

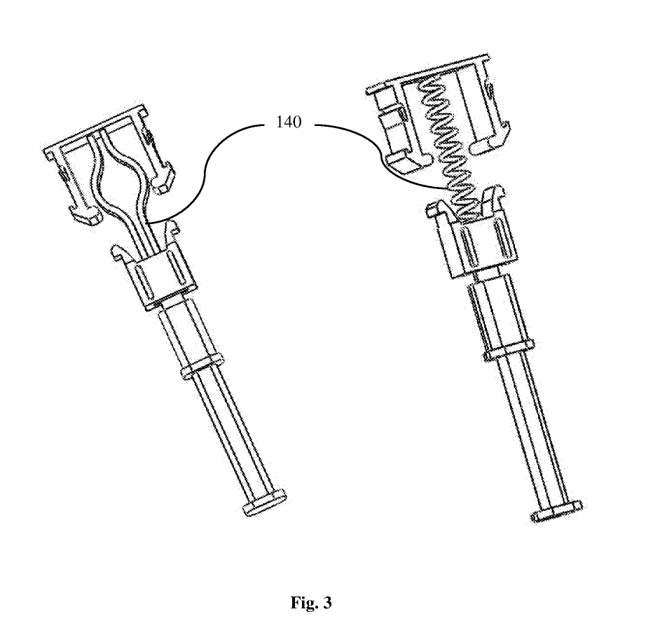

The present invention provides a blood lancet unitarily moulded with an elastic member for providing the required actuation force. In the preferred embodiment 100, which is shown in FIG. 1 and FIG. 2, the present invention involves a needle component 120, which further comprising a forward end 122 and a rearward end 124, an elastic member 140, and a detachable safety cap 160. The needle component 120 can be made of several materials such as polymers, metals and ceramics. In the preferred embodiment, polymer poly-etherimide is used for making the needle component 120 by injection moulding. The elastic member 140 is unitarily moulded on the rearward end 122 of the needle 120. Similarly, the detachable safety cap 160 is unitarily moulded on the forward end 124 of the needle component 120.

If the needle component 120 is made of metals or ceramics, the unitary moulding process involved is insert moulding, in which the needle component 120 is inserted into a mould and a polymer resin is injected over the needle component 120 to form the elastic member 140 and the detachable safety cap 160.

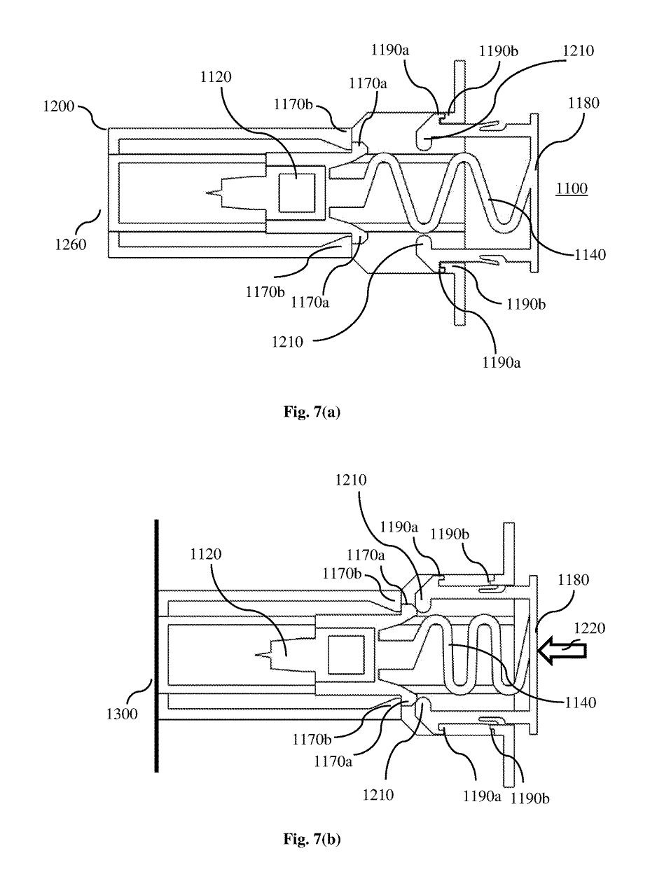

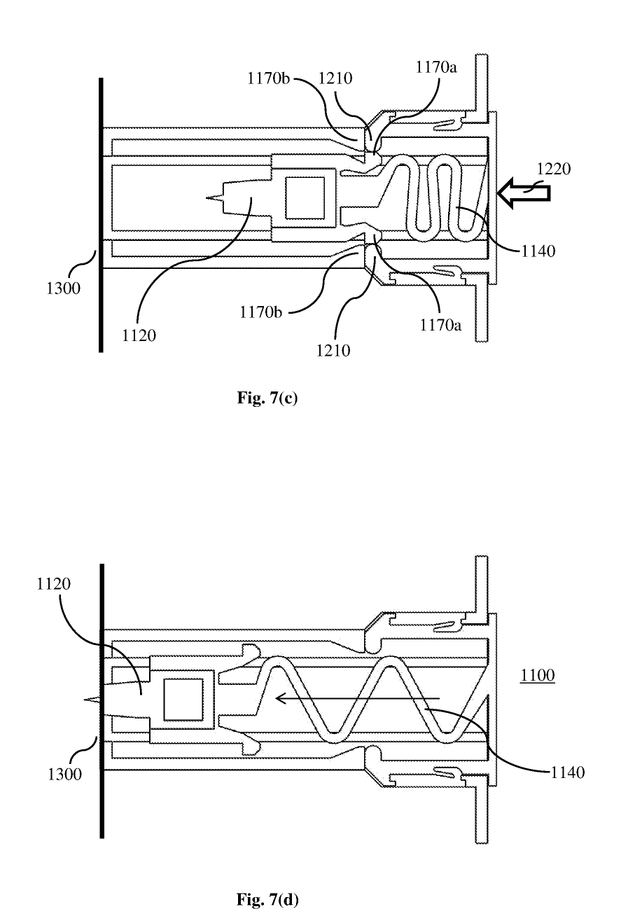

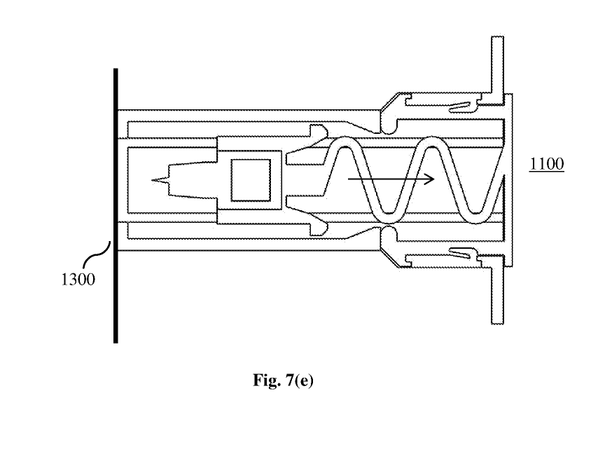

If the needle component 120 is made of polymer resin, such as poly-etherimide, the unitary moulding process involved is twin-shot injection moulding, or more commonly known as ‘two-colour’ moulding. The preferred embodiment blood lancet 100 is preferably made by twin-shot injection moulding wherein the needle component 120 is first moulded with a first material, namely poly-etherimide, and subsequently the elastic member 140 and the detachable safety cap 160 is over-moulded with a second material, namely polyacetal (POM), polyethylene, polypropylene, and so on, onto the rearward end 122 and the forward end 124 of the needle component 120 respectively. The detachable safety cap 160 protects the needle tip and will be pulled off to expose the needle at the point of use. The elastic member 140 will provide the required spring force or actuation force when it is charged and then dis-charged, as described in FIG. 7.

A preferred method for manufacturing the blood lancet 100 is by twin-shot injection moulding. Materials used for injection moulding the blood lancet 100 should be bio-compatible and gamma-irradiation stable. In addition, the polymer for moulding the needle component 120 should have sufficient impact and flexural strength. Particularly important is that the glass transition temperature (the temperature that a polymer starts to soften) should be higher than the melting temperature of the second plastic resin to prevent distortion or deformation of the plastic tip. One identified material that has all the characteristics is polyether-imide (PEI), which has a glass transition temperature of 220° C.

On the other hand, the second polymer for moulding the elastic member 140 and the detachable safety cap 160 does not require high impact and flexural strength but has to have a low melting temperature so that the heat will not affect the shape of the needle tip during the second moulding process. Another important condition is that there should be no chemical or covalent bonding present between the first material PEI and the second material, otherwise the detachable safety cap 160 cannot be removed to expose the needle tip. One identified polymer is high density polyethylene (HDPE), which has a melting temperature of 130° C. and does not have covalent bonding after the over-moulding process. The low melting temperature is good for a stable moulding process because under normal circumstances the polymer melt is normally raised to higher temperature to enhance flowability of the polymer melt. The wide gap between the melt temperature of HDPE and the glass transition temperature of PEI provides a good range of process window under which the over-moulding can be operated.

FIG. 3 shows two other designs that the elastic member 140 can take. The geometry and the shape of the elastic member 140 are not crucial so long as it provides the required actuation force when it is charged and dis-charged.

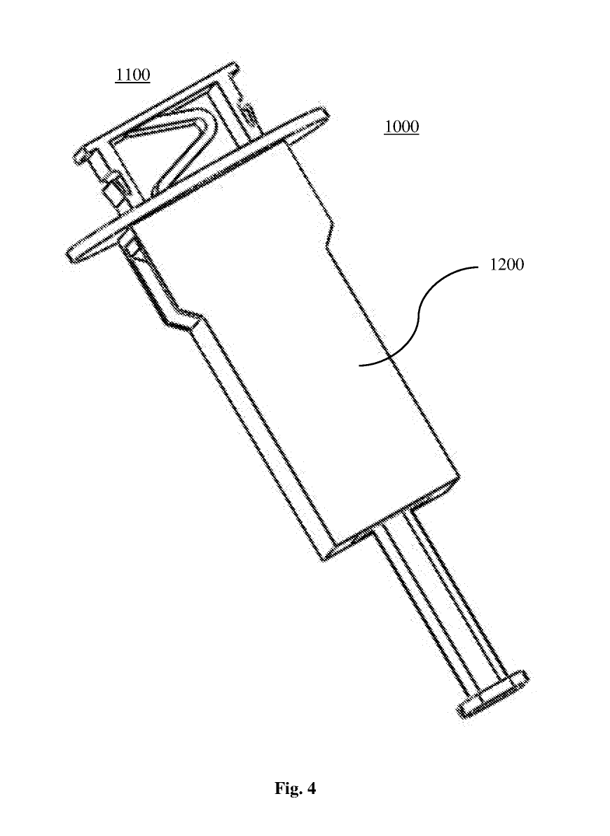

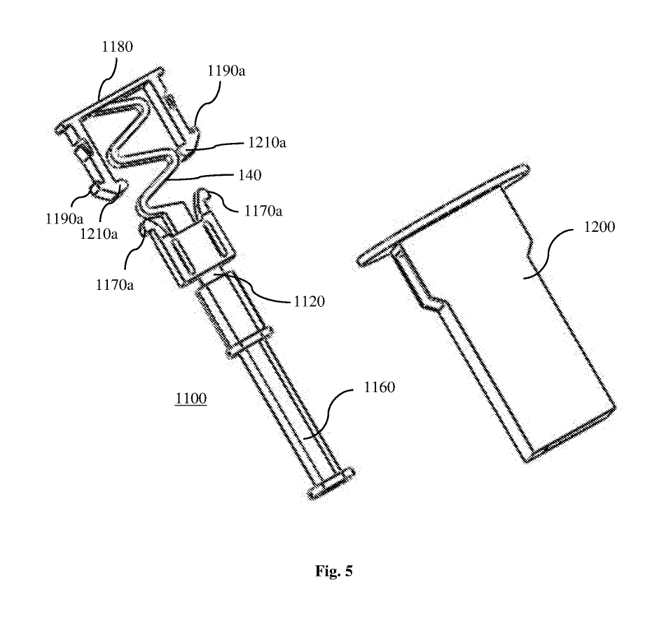

FIGS. 4 and 5 show the second preferred embodiment which is a safety lancet 1000, which comprises a blood lancet 1100 and a holder 1200. FIG. 4 shows a perspective view of the safety lancet 1000 in which the blood lancet 1100 is assembled in the holder 1200. FIG. 5 shows the exploded view of the safety lancet 1000 which comprises a blood lancet 1100 and a holder 1200. The blood lancet 1100 further comprises a needle component 1120 having a forward end and a rearward end, an elastic member 1140 and a detachable safety cap 1160.

### Assembly of the Second Preferred Embodiment

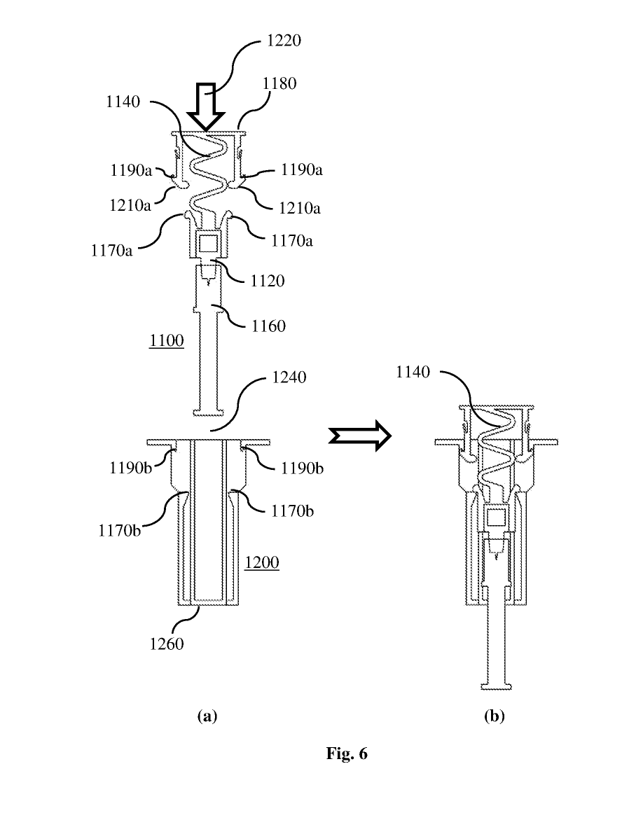

FIG. 6 shows the section view of the safety lancet 1000 during its assembly process. For the assembly of the safety lancet 1000, the blood lancet 1100 and the holder 1200 are provided. The blood lancet 1100 comprises a needle component 1120 having a forward end and a rearward end, an elastic member 1140 disposed at the rearward end, a detachable safety cap 1160 disposed at the forward end, a pair of latch hooks 1170a attached to the needle component 1120, a U-shape button 1180 having a pair of arms, each arm further comprises a hammer 1210a and a reverse hook 1190a at the end of the arm, wherein all the components, i.e. the elastic member 1140, the detachable safety cap 1160, the pair of latch hooks 1170a, the U-shape button 1180, the pair of hammers 1210a and the pair of reverse hooks 1190a, are unitarily moulded on the needle component 1120.

On the other hand, the holder 1200 is a substantially longitudinal hollow body which is designed to receive the blood lancet 1100. For example, the holder's 1200 inner surface is moulded with a pair of latch stoppers 1170b for engaging the latch hooks 1170a and a pair of reverse stoppers 1190b for engaging the reverse hooks 1190a, wherein the latch hooks 1170a and reverse hooks 1190a are unitarily moulded on the blood lancet 1100. The holder 1200 also comprises a proximal opening 1240 and a distal opening 1260.

During the assembly process, a blood lancet 1100 is inserted into the holder 1200 via its proximal opening 1240 by applying compression force 1220 on the U-shape button 1180. The insertion will be first resisted by the pair of hammers 1210a at the end of the arms of the U-shape button 1180, after which the hammers 1210a will be overcome by the compression force 1220 and are pushed inward so that the blood lancet 1100 can go into the holder 1200. The blood lancet 1100 will continue to be pushed forward until the latch hooks 1170a are stopped by the latch stoppers 1170b on the holder 1200. At this moment, the elastic member 1140 is slightly compressed (or charged). Finally, the compression force 1220 is removed and the U-shape button 1180 will retract slightly due to the relaxation of the elastic member 1140 and the blood lancet 1100 is now locked by the latch stoppers 1170b and the reverse stoppers 1190b. In this final configuration, the blood lancet 1100 is firmly held in the holder 1200 but the elastic member 1140 is not charged. Pre-charging the elastic member 1140 will subject the elastic member 1140 to material creeping (a common phenomenon for plastic material), which sees the elastic member 1140 lengthening over time and losing the elasticity for providing actuation force.

### Operations of the Second Preferred Embodiment

FIGS. 7(a)-(e) show the operation of the safety lancet 1000. Firstly, the detachable safety cap 1160 is removed as shown in FIG. 7(a). At this stage, the blood lancet 1100 is firmly held in the holder 1200 and the elastic member 1140 is only slightly charged (or compressed). To use the safety lancet 1000, a user holds the device in between his index and middle fingers and points the device at a skin site 1300.

Next, as shown in FIG. 7(b), the user uses his thumb to exert a compression force 1220 onto the U-shape button 1180 which pushes the U-shape button 1180 forward and charges (or compresses) the elastic member 1140. The compression or charging of the elastic member 1140 is achieved because the blood lancet 1100 is prevented from moving forward due to the engagement of the latch hooks 1170a and the latch stoppers 1170b.

Subsequently, as shown in FIG. 7(c), the hammers 1210 on the arms of the U-shape button 1180 make contact with the latch hooks 1170a and push them inward, thereby releasing the latch which is formed by the latch hooks 1170a and the latch stopper 1170b.

As a result, as shown in FIGS. 7(d) and 7(e), the charged elastic member 1140 releases its stored potential energy and actuates the needle component 1120 forward. The needle component 1120 travels linearly at 1 m/s-6 m/s and penetrates the skin site 1300, making an incision on the skin, then quickly retracts back to the holder 1200 at a retraction speed similar to the penetration speed.

1. A blood lancet for capillary blood sampling, comprising
a. A needle component having a forward end and a rearward end,
b. A detachable safety cap unitarily moulded to the forward end, and
c. An elastic member unitarily moulded to the rearward end,
wherein the needle component is made of poly-etherimide.

2. (canceled)

3. (canceled)

4. (canceled)

5. (canceled)

6. (canceled)

7. A blood lancet in claims 1, wherein the detachable safety cap and the elastic member are made of plastic resin such as polyacetals (POM), polyethylene or polypropylene.

8. A blood lancet for capillary blood sampling, comprising
a. A needle component moulded with a first plastic resin, the needle component having a forward end and a rearward end,
b. A detachable safety cap unitarily moulded with a second plastic resin to the forward end, and
c. An elastic member unitarily moulded with a second plastic resin to the rearward end,
Wherein the melting temperature of the second plastic resin is lower than the glass transition temperature of the first plastic resin.

9. A blood lancet in claim 8, wherein the elastic member further comprising a U-shape button having two arms which is unitarily moulded to the elastic member.

10. A safety lancet for capillary blood sampling, comprising
a. A holder, and
b. A blood lancet, the blood lancet further comprising
i. A needle component having a forward end and a rearward end,
ii. A detachable safety cap unitarily moulded to the forward end, and
iii. An elastic member unitarily moulded to the rearward end,
wherein the needle component is made of poly-etherimide.

11. (canceled)

12. (canceled)

13. (canceled)

14. (canceled)

15. (canceled)

16. A safety lancet in claims 5, wherein the detachable safety cap and the elastic member are made of plastic resin such as polyacetals (POM), polyethylene or polypropylene.

17. (canceled)

18. (canceled)

19. (canceled)
